(19) United States
(12) Patent Application Publication
(10) Pub. No.: US 2007/0098631 A2
(43) Pub. Date: May 3, 2007

(54) DIAGNOSTIC COMPOUNDS COMPRISING A SCAFFOLD COUPLED TO A SIGNAL ENTITY FOR MEDICAL IMAGING DIAGNOSTIC

(75) Inventors: Claire COROT, Lyon (FR); Marc PORT, Deuil La Barre (FR); Thierry GAUTHERET, Bois Le Roi (FR); Xavier WILLIARD, Epone (FR)

Correspondence Address: BIRCH STEWART KOLASCH & BIRCH, P. O. BOX 747, FALLS CHURCH, VA, 22040-0747, US

(73) Assignee: GUERBET, Villepinte (FR)

(21) Appl. No.: 10/833,282

(22) Filed: Apr. 28, 2004

(51) Int. Cl. A61K 51/00; A61K 49/00; C07F 5/00

(52) U.S. Cl. 424/1.11; 534/11; 534/15; 424/9.36; 977/927

(57) ABSTRACT

The present invention concerns a diagnostic compound of formula
(SCAFFOLD)ₙ₁-(LINKER)ₙ₃-(SIGNAL)ₙ₂-(M), (E)
wherein SCAFFOLD is chosen in table 2: 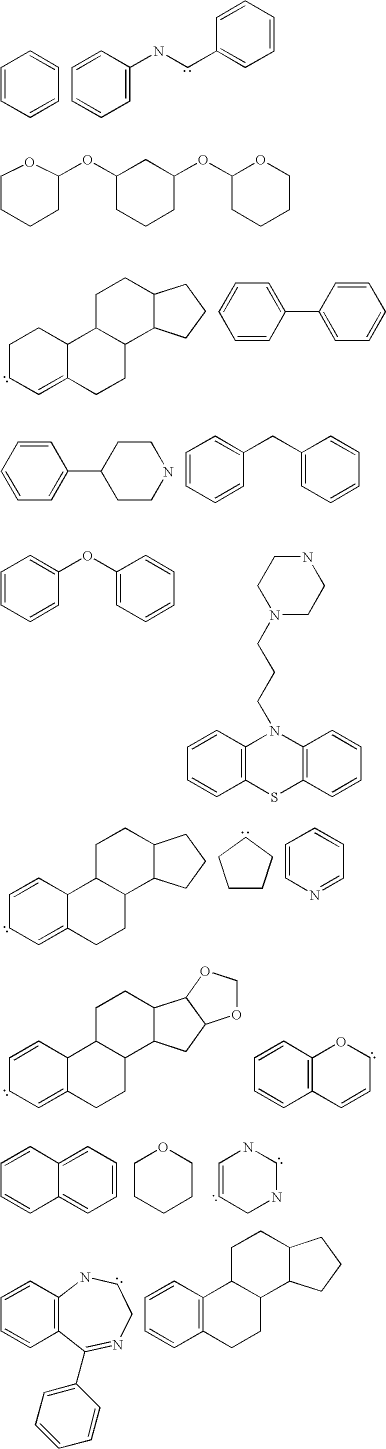 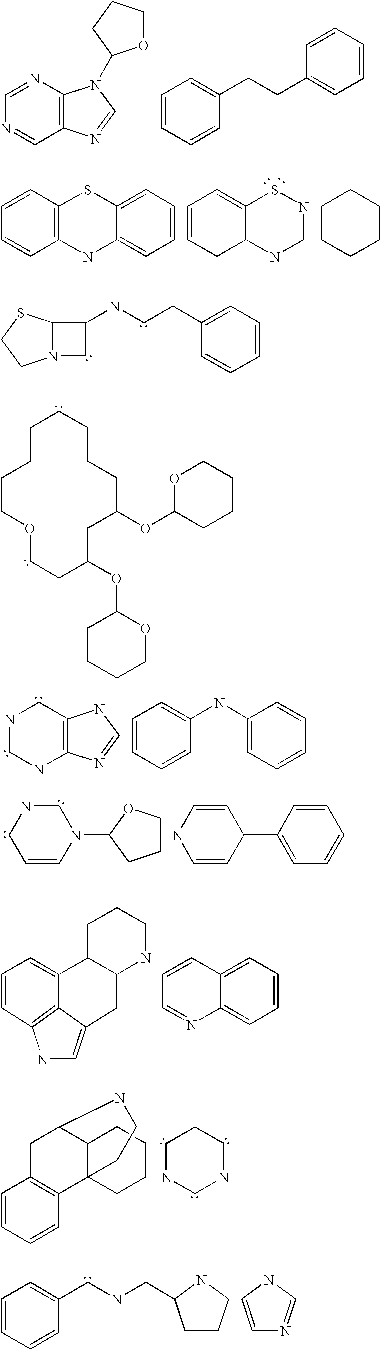 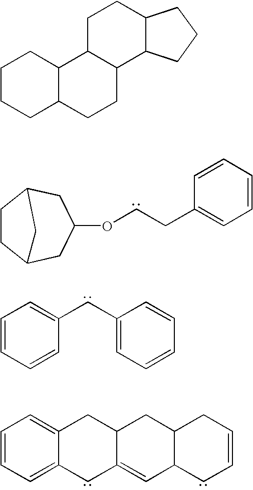 namely Biphenyl; Arylpiperidine; Arylpiperazine; 1,4 dyhydropyridine; Dihydropyrimidone; 1,4 benzodiazepine-2-one; 1,5 benzodiazepine-2-one; 1,4-benzodiazepine-2,5 diones; pyrrolo2,1-c 1,4 benzodiazepines 5,11 diones; 1,4 benzothiazepine-5-ones; 5,11-dihydro-benzo pyrido 3,2b 1,4 diazepine-6-ones Benzopyrane; Chromone; Benzopyranone; Coumarine, pyranocoumarine Benzopiperazinones; Quinazolinone; Quinazolindione; Quinoxalinone Imidazoquinoxaline; indole; Benzimidazole Benzofurane Benzothiophene; SIGNAL is a signal entity for medical imaging diagnostic, and pharmaceutically acceptable salts thereof.

The invention relates to new compounds and compositions for the imaging diagnostic of pathologies, namely for cardiovascular, cancerous and inflammatory diseases.

These compounds are contrast agents useful namely in the fields of magnetic resonance imaging MRI, nuclear medicine, X-ray, ultrasounds, optical imaging.

These compounds comprise at least a targeting entity linked to at least a signal entity. A targeting entity is capable of targeting at least one marker of a pathologic state and/or area, for instance enzymes or cellular receptors that are over or under expressed in a pathologic state and/or area. These compounds are called specific compounds, the targeting entity being called biovector.

Numerous signal entities are already known, such as linear or macrocyclic chelates of paramagnetic metal ion for MRI and of radionucleides for nuclear medicine. Such chelates are described in the documents EP 71 564, EP 448 191, WO 02/48119, U.S. Pat. No. 6,399,043, WO 01/51095, EP 203 962, EP 292 689, EP 425 571, EP 230 893, EP 405 704, EP 290 047, U.S. Pat. No. 6,123,920, EP 292 689, EP 230 893, U.S. Pat. No. 6,403,055, WO 02/40060, U.S. Pat. No. 6,458,337, U.S. Pat. No. 6,264,914, U.S. Pat. No. 6,221,334, WO 95/31444, U.S. Pat. No. 5,573,752, U.S. Pat. No. 5,358,704. Chelates commonly used are DTPA, DTPA BMA, DTPA BOPTA, DO3A, HPDO3A, TETA, DOTA, PCTA and their derivatives. The signal is measured in MRI by the relaxivity in water which is in the order of 3 to 10 mM-1s-1 Gd-1. Some specific compounds are known in the prior art.

The applicant has studied new specific contrast products, namely for MRI and/or nuclear medicine, by using biovectors that have chemical structures, and more precisely chemical scaffolds, that are known for their biological activity but which use for the imaging diagnostic field was not known nor suggested by the prior art. Indeed, the applicant has focused on the fact that among the 5000 main therapeutic drugs, only around 30 scaffolds (called major scaffolds and represented in table 1), are common to almost 50% of these drugs.

Thus the invention relates to compounds comprising scaffolds which biological targeting property is established since they are known as drugs, but which use as target entity of a specific contrast product was not known.

Among these major scaffolds and their derivatives, the applicant has studied and prepared two types of scaffold to be used in the imaging diagnostic field when they are coupled to at least a signal entity:
scaffolds that are known to be efficient in the therapeutic field that have the ability to target at least a ligand indicative of a pathologic state or area, and for which the therapeutic corresponding drugs are not toxic in the therapeutic field
scaffolds that are known to have the ability to target a ligand indicative of pathologic state or area, that may be toxic in the therapeutic field, but that are not toxic in the imaging diagnostic field, in view of the dose and rate of administration of the contrast product.

The invention also relates to derivatives of these scaffolds coupled to signal entities. These derivatived scaffolds are either already known in the prior art such as described in table 3 above, or may be obtained after a study of structure activity relationship.

Considering the higher sensibility of the nuclear medicine compared to MRI, the chelate can be less complex for the nuclear medicine. The choice of the biovector entity (the scaffold), the signal entity, and the linker between these two entities is made appropriate for an efficient use in the imaging diagnostic.

The efficiency of the selected scaffolds as biovector of a specific contrast product is tested according to appropriate practices in vitro, on in vivo biological models and according to standard procedures of imaging known by the one skilled in the art.

In an aspect the invention relates to new compounds of formula:
(SCAFFOLD)ₙ₁-(LINKER)ₙ₃-(SIGNAL)ₙ₂-(M), (E)
wherein
1) SCAFFOLD is chosen among the SCAFFOLDS of table 1 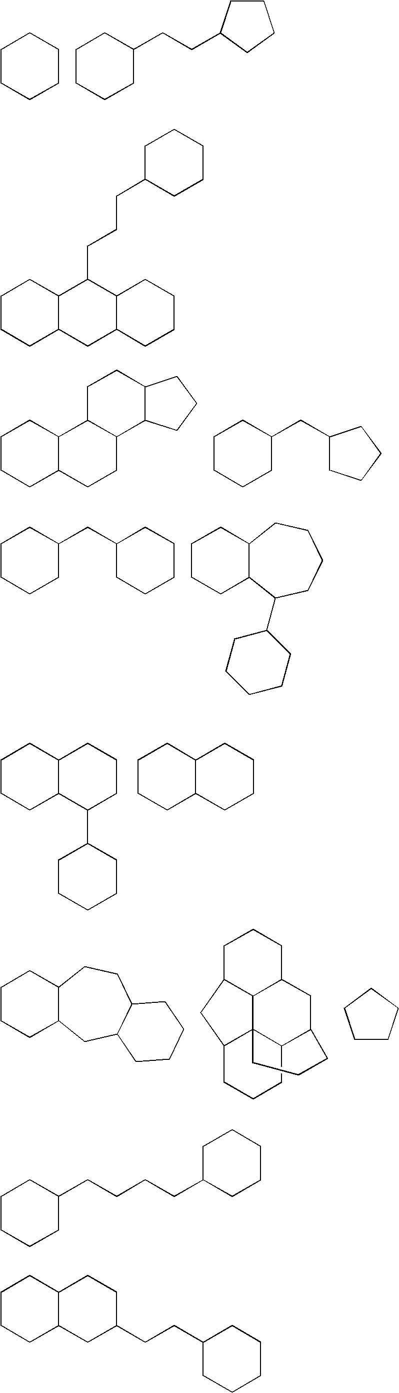 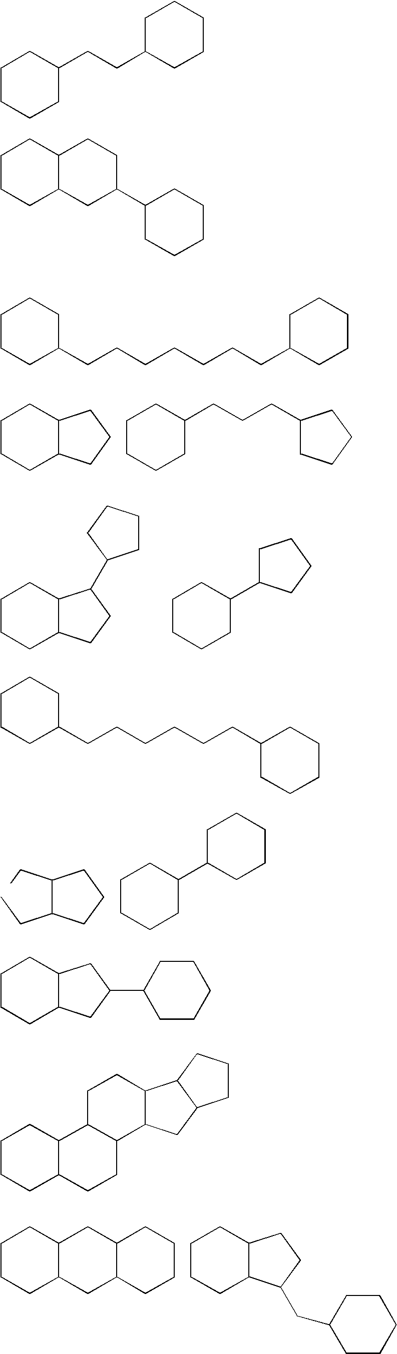 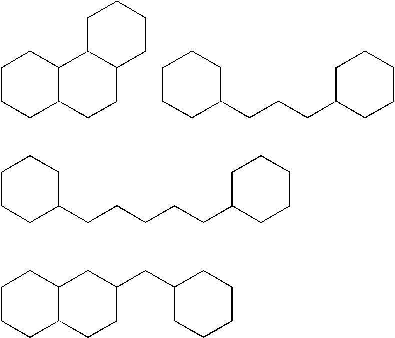
2) SIGNAL is an entity capable of generating a signal in medical imaging
3) LINKER is a chemical link between a scaffold and a signal entity, and the pharmaceutically acceptable salts thereof.

According to a preferred embodiment, SCAFFOLD is chosen among the scaffolds of table 2.

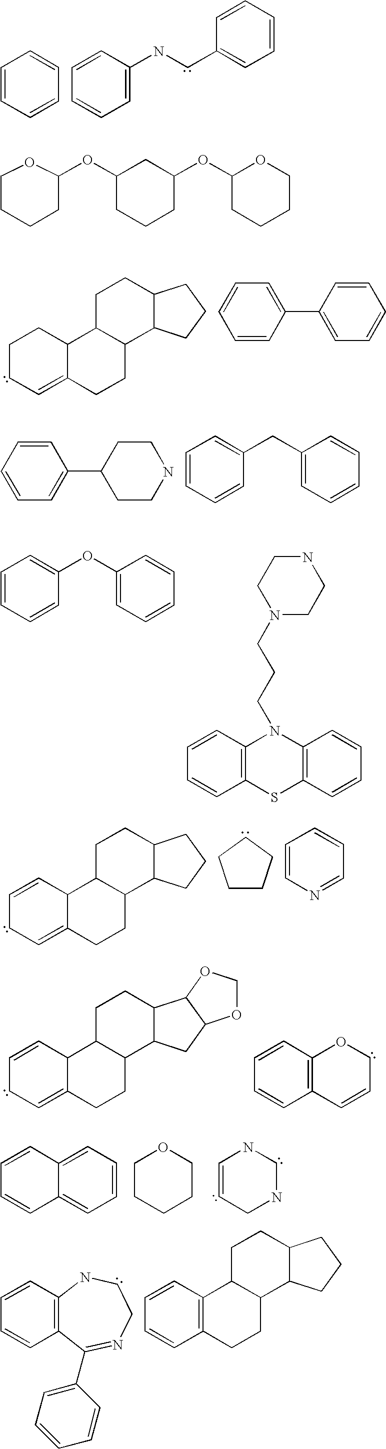

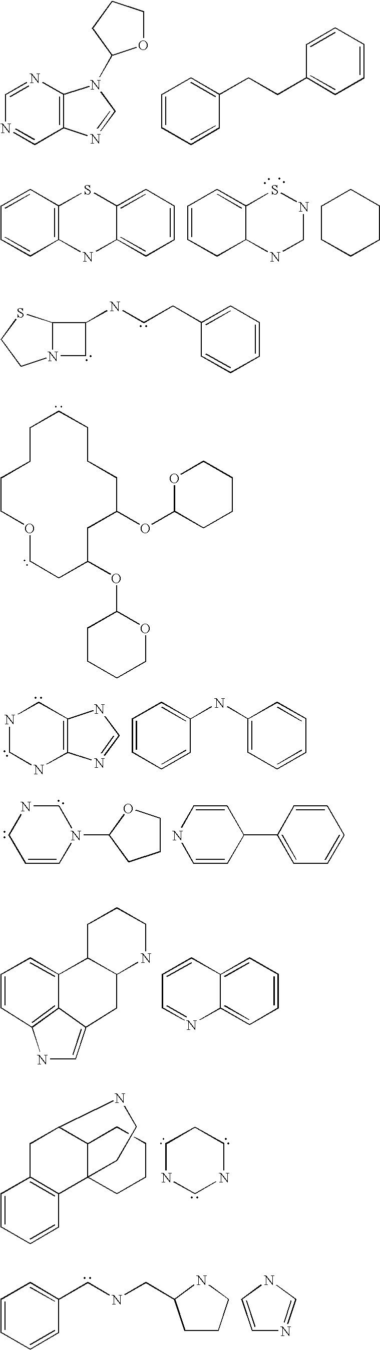

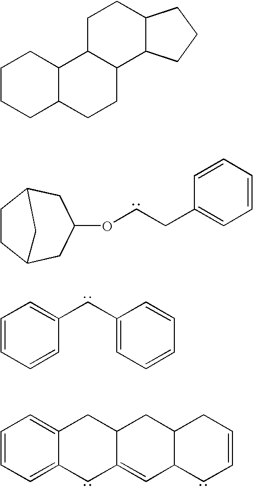

According to an embodiment, SCAFFOLD is chosen among the scaffolds of following table 3. Main activities and examples of method of manufacturing of such scaffolds and derivates scaffolds (typically obtained by combinatorial synthesis) are reminded namely in Chemical reviews, 2003, vol 103 no 3,893-930 incorporated therein by reference.

| | | Examples of biological |
|---|---|---|
| Structure | Scaffold and derivatives | activity known |
| Phenyl substituted | Biphenyl | Antithrombotic, |
| monocycles | | antiinflammatory, |
| | | antitumoral, |
| | | antiarhythmic, |
| | | antiatherosclerotic |
| | Arylpiperidine | targets neurokinin |
| | | receptors |
| | Cis-(2S,3S)-pipéridine | (pathologies: migraine, |
| | (8, 9, 10, 20 to 24) | arthritis, asthma . . . ) |
| | | inhibitor of |
| | | neuropeptides, |
| | | somatostatin |
| | | (antitumoral), serotonin |
| | | receptor |
| | Arylpiperazine | cardivascular diseases, |
| | (27) | arrhytimia, antiaggregant, |
| | | endothelin antagonist |
| | | antitumoral |
| | 1,4 dyhydropyridine | Antihypertension, anti- |
| | (28, 29, 40) | arrhytimia, antitumor, |
| | | antiinflammatory |
| | Dihydropyrimidone | Antihypertension, |
| | (45, 49, 53) | anticancer, cardiovascular |
| | | (platelets) |
| Cycles linked [7, 6] | 1,4 benzodiazepine-2-one | Nervous central system |
| Benzodiazepine | (61, 71, 75, 82) | Antagonist for |
| | | neurokinine, agonist for |
| | | opioid receptor, |
| | | cholecystikinine |
| | | receptor . . . |
| | | Tyronise Kinase inhibitor |
| | 1,5 benzodiazepine-2-one | Converting enzyme |
| | (62, 83, 84, 89, 91) | inhibitors |
| | 1,4-benzodiazepine-2,5 diones | Antitumoral, antogonist |
| | (63, 95, 100, 103) | for CCK receptor, target |
| | pyrrolo2,1-c 1,4 benzodiazepine | glycoproteins GpIIb-IIIa |
| | 5,11 diones | |
| | (65) | |
| | 1,4 benzothiazepine-5-ones | Converting enzyme |
| | (64, 109) | inhibitors |
| | | Antitumor |
| | 5,11-dihydro-benzo pyrido 3,2b | |
| | 1,4 diazepine-6-ones | |
| | (66, 110, 116) | |
| Cycles linked [6, 6] | Benzopyrane | Antitumoral |
| | (117, 125, 132) | Antiinflammatory |
| | Chromone | Inhibitor of protein |
| | (118) | kinases |
| | Benzopyranone | Antihypertensive |
| | (135, 140, 146, 154, 155) | anticancerous |
| | Coumarine, pyranocoumarine | Anticoagulant |
| | (119, 156, 160, 161, 162, 169, 170) | Anticancer |
| Cycles linked [6, 6] | Benzopiperazinone | |
| Quinoxaline/quinazolines | (181, 187, 203, 200, 192) | |
| | Quinazolinone | Cardiovascular and |
| | (182, 184, 210, 211, 217, 225, 233) | antiinflammatory activity |
| | Quinazolindione | Activity on nervous |
| | (185, 238) | central system |
| | | Coagulant (fibrinogen |
| | | receptors antagonists) |
| | Quinoxalinone (183) | |
| | Imidazoquinoxaline | Activity on nervous |
| | (186, 244, 245, 246, 247, 251, 252, | central system |
| | 253, 256, 261, 265) | |
| Cycles linked [5-6] | Indole | Activity on nervous |
| | (266, 270, 271, 272, 273, 277, 283, | central system, |
| | 288, 293, 299) | antiinflammatory, |
| | | cardiovascular activity |
| | | (target somatostatin |
| | | receptors, thrombin) |
| | Benzimidazole | Anticancer, |
| | (267, 305, 306, 307) | antihistaminic, |
| | | antiartythmic, targets |
| | | integrins |
| | Benzofurane (268, | Cardiovascular |
| | 308, 309, 313, 318, 332, 336) | (hypertension, platelet |
| | | aggregation), antimitotic |
| | Benzothiophene (269, 337, 342) | Antimitotic, inhibitor of |
| | | serine proteases |

The number in brackets are those attributed in the scaffolds depicted in Chemical reviews, 2003, vol 103 no 3, incorporated by reference. This document teaches the synthesis of these scaffolds of table 3. Advantageous scaffolds are the following and their derivatives known in the art.

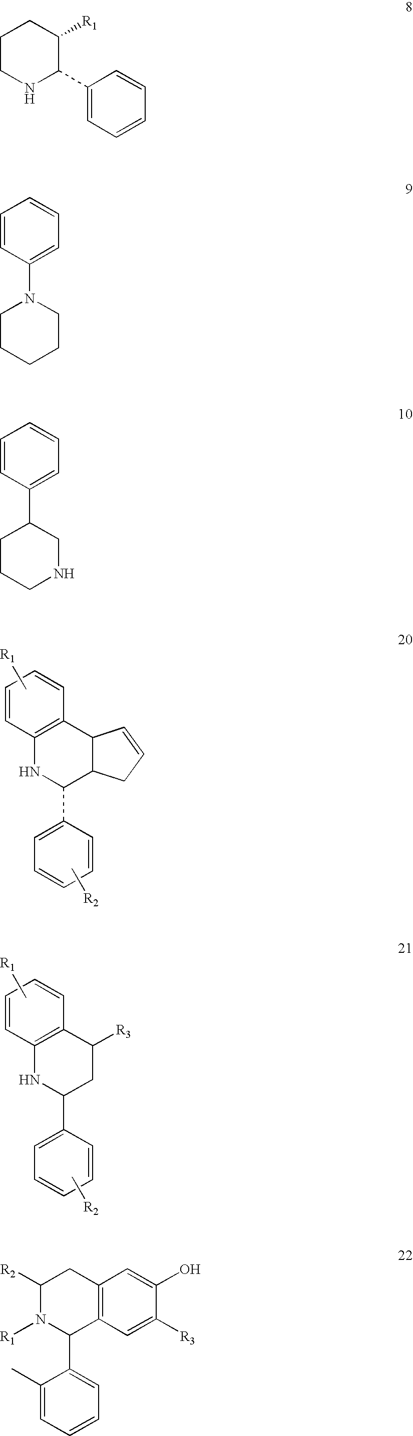

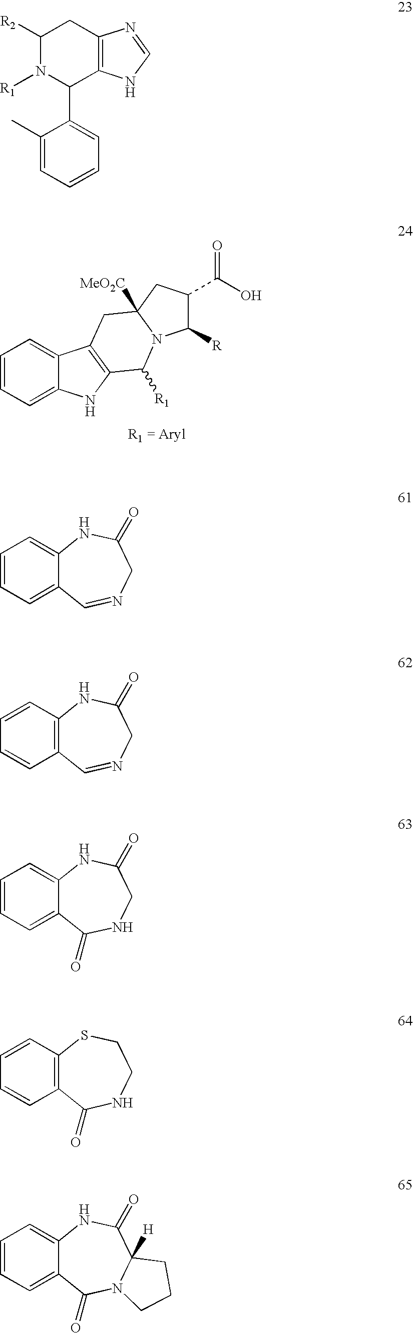

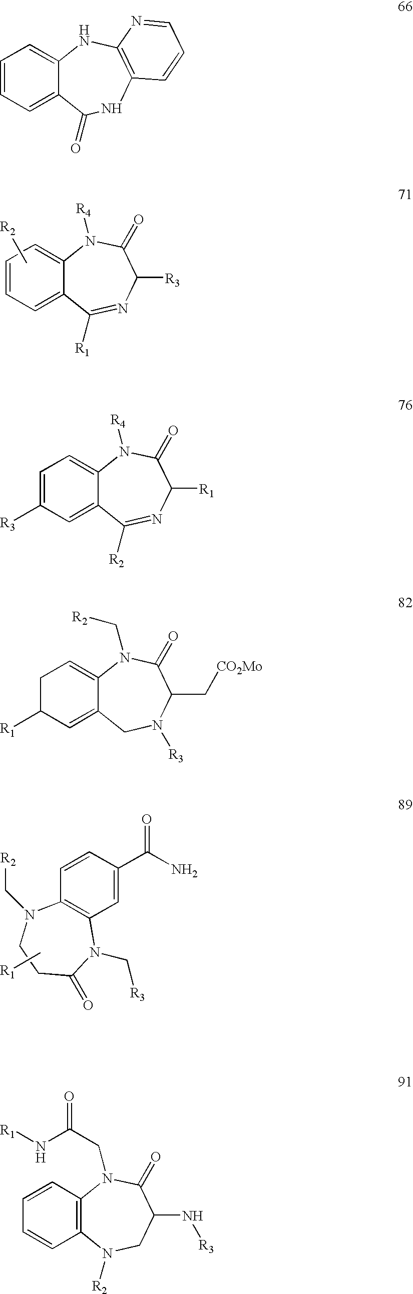

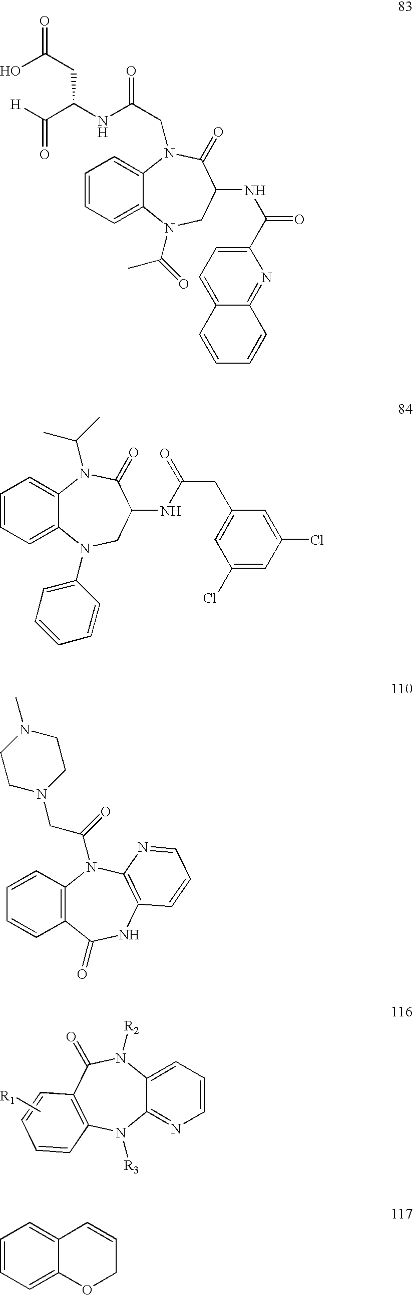

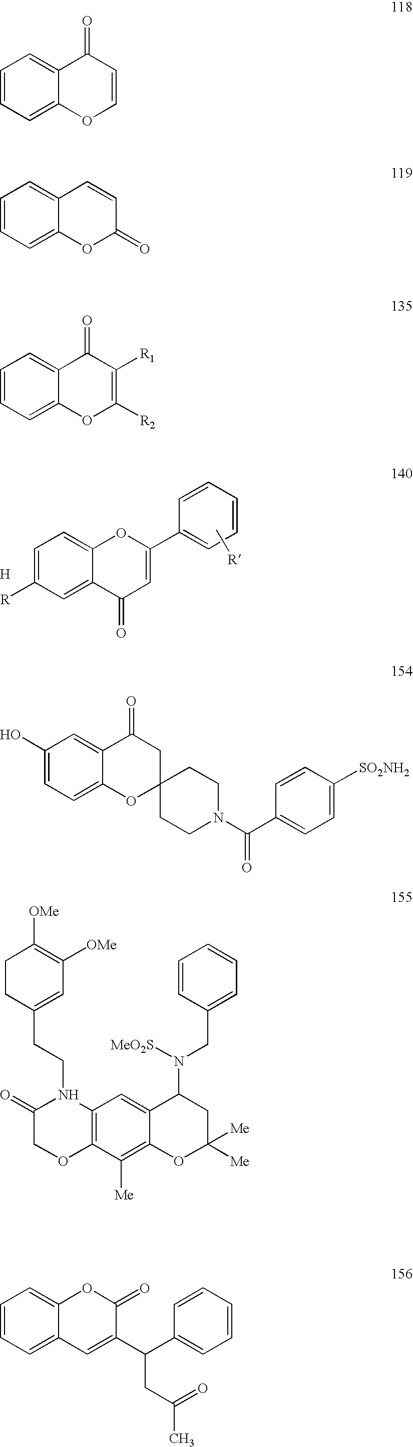

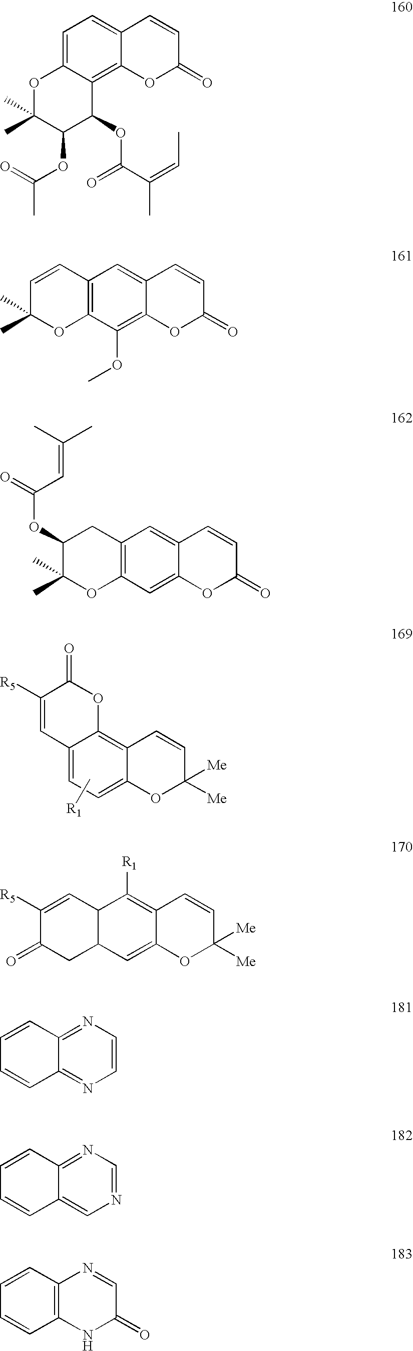

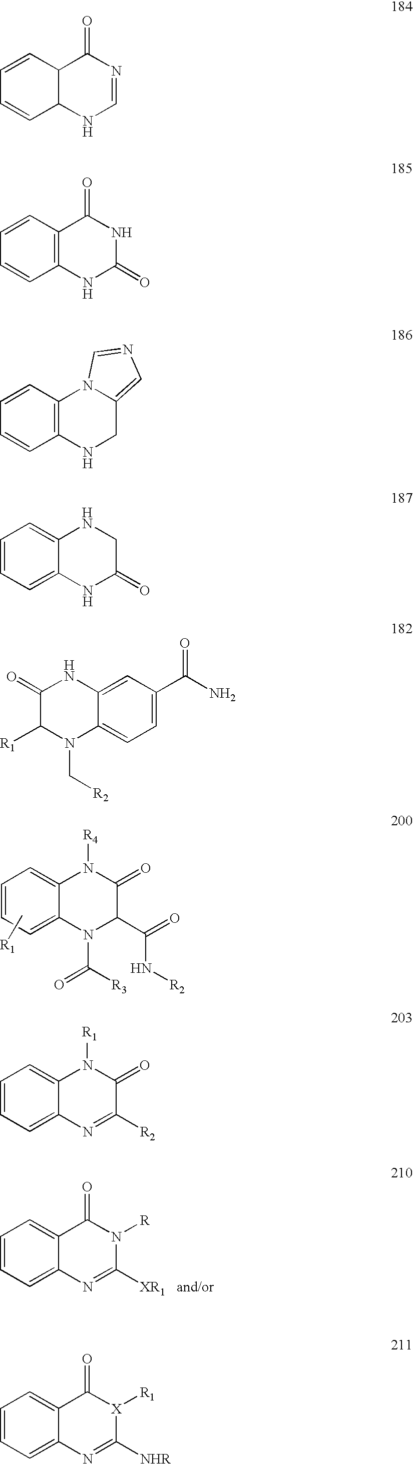

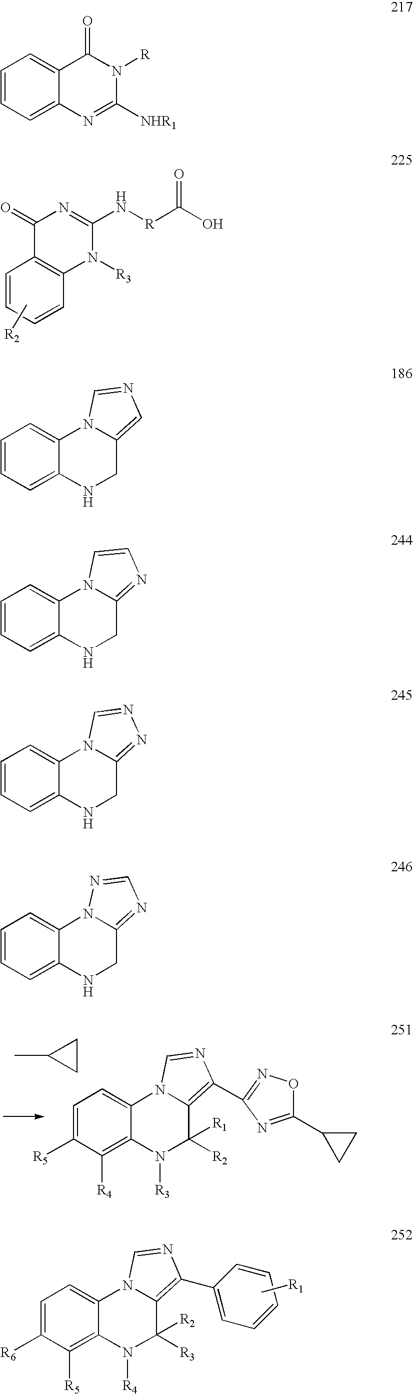

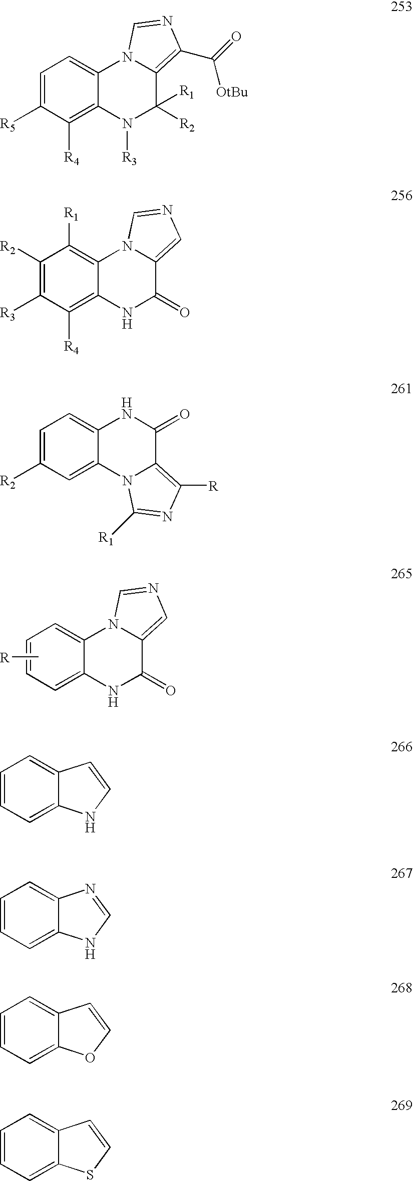

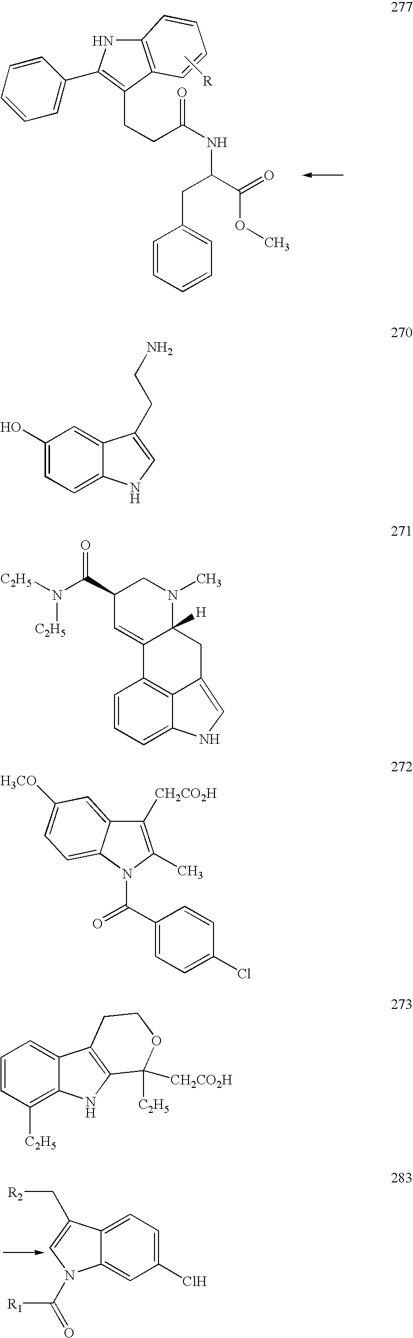

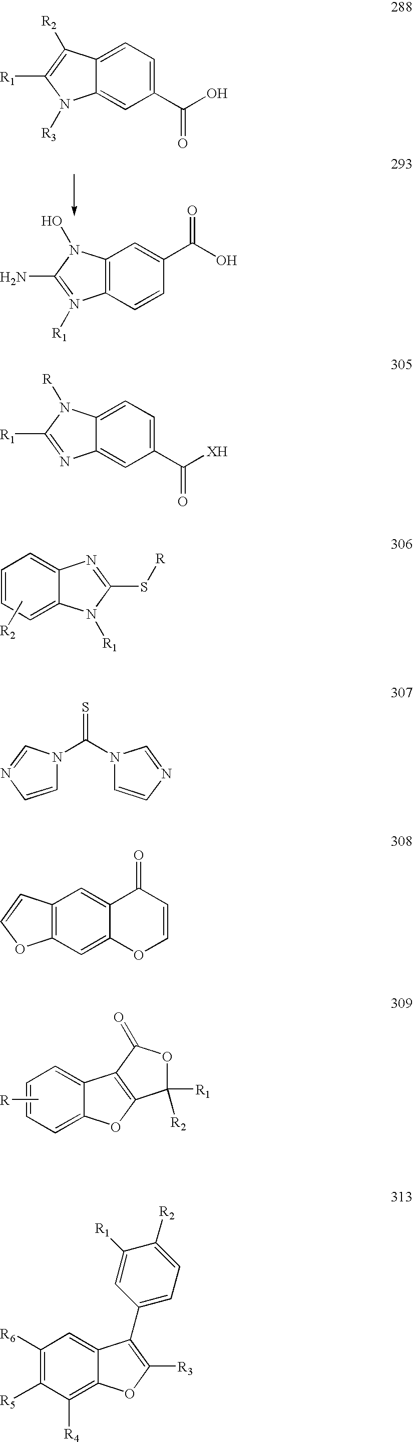

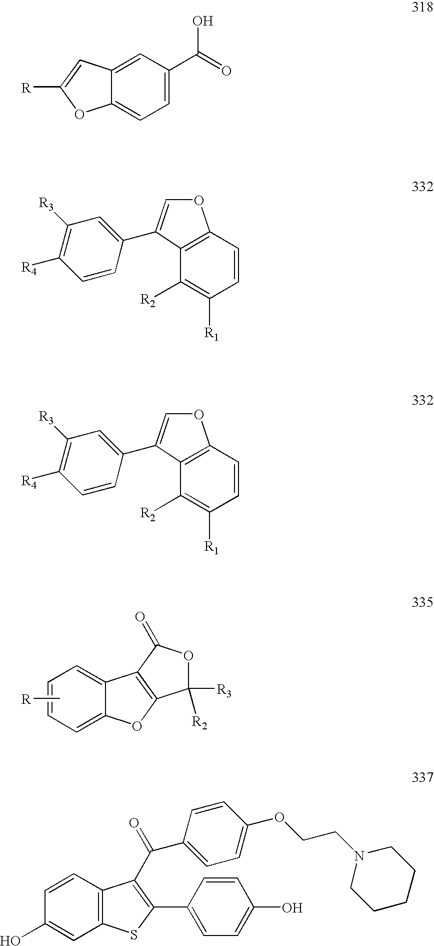

The tables 1 to 3 are not exclusive in terms of chemical structures and of biological activities. The applicant methodology includes identifying scaffolds with substantial probability of efficiency in imaging diagnostic, and using these scaffolds to target ligands that are known as or that are presumably appropriate markers of a pathologic state or area.

For instance, in order to construct candidate compounds targeting tyrosine kinases that are known to be associated to pathological states, the applicant teaches to identify and prepare chemical scaffold(s) and/or scaffold derivatives that are known to target tyrosine kinases and to bind the scaffold to a signal entity. Such methodology may include a step of optimising the scaffold entities, with the help of study of structure activity relationship, and then to do the coupling. Such structure activity studies are described for instance for protein tyrosine phosphatase associated to a diabetic state (see Journal of Medicinal Chemistry, 2003, vol 46, no 22) and for MMP inhibitors (see J. Bioorg. Med. Chem. lett 13, 2003, 1487-1490 that describe scaffold quinoline and pyrazolopyridine). Such methodology applies to the main biological targets in the therapeutical field, for instance for COX inhibitors, in particular COX2.

Besides, it is now known in the art (see Annual reports in medicinal chemistry—37-page 194) that most of the launched drugs target a limited number of proteins (238 protein targets, such as rhodopsin-like GPCR, nuclear hormone receptor, serine protease, monoamine oxidase). The applicant teaches to identify scaffolds able to target such major proteins, and to couple them with signal entities. It is also described herein that major targets of interest are biological targets that are key metabolic targets in biological pathways, such as enzymes that are involved in several different metabolic pathways associated with pathologic states; such targets are also in the frame of the invention.

Further, the methodology implies to functionalise the scaffolds so that they can be coupled efficiently to the signal entities. The scaffolds either already comprise a chemical function such as amino or carboxy that can react with the signal entity, or are chemically modified so that they comprise such coupling function. Similarly, the signal entities are chemically prepared for an appropriate coupling. Typically the compound (E) comprises at least one LINKER such as a PEG group or a peptidic or peptidomimetic linker, in order to avoid misappropriate interaction between the scaffold and the signal entity. Many examples of LINKER are described for instance in WO 01/60416 incorporated by reference. Typically the scaffolds target an entity that is over or under expressed in the pathologic area compared to the normal one. Their target may be intracellular, membranar, or extracellular. Further to the scaffolds described herein, the biovectors may have very different types of conformations and behaviors. Several scaffolds may be associated in a same diagnostic compound, for instance different scaffolds that are to target a same pathologic tissue or type of cells indicative of a same pathology. Scaffolds may be linked together, their association being coupled to a signal entity. The scaffolds may be coupled to a same signal entity at different sites of anchoring that are present on the same signal entity such as a nanodroplet or a metallic nanoparticle or a chelate. One or several scaffolds may be coupled to an other biovector such as a biological polymer, for instance a polypeptide, a protein, a polysaccharide. The signal entities may be coupled to the scaffolds and/or to the other biovector. A scaffold derivative chemically modified may comprise several sites of anchoring a signal entity such as a chelate. A scaffold derivative may comprise different regions of different chemical affinity, for instance hydrophobic or hydrophilic domains so that they can be used in different ways.

An other way to identify and screen scaffolds of strong interest is to use the so called SOSA method (Selective Optimization of Side Activities) described in Journal Chemistry, vol 47, no 6, 2004, 1303-1314. This approach, originally applied to the scaffolds by the applicant into the imaging diagnostic field, consists in testing scaffold and derivatives that are known for certain therapeutical applications, for other new diagnostic applications. This allows to test a limited number of drug scaffolds that have known safety and bioavailability in humans. Once the scaffolds presumably efficient in the imaging diagnostic field selected, the hits are optimized (traditional, parallel or combinatorial chemistry) in order to increase the affinity for new potential targets that are searched. The applicant utilises the fact that drugs/scaffolds in humans interact with more than one target/receptor. Thus the applicant methodology includes to test known scaffolds for new diagnostic applications after a coupling with a signal entity. Examples are given in the following table 4.

| | | Modified | Activity newly |
|---|---|---|---|
| Scaffold | Known activity | scaffold | identified |
| Dihydropyridines | Target calcium | 14 | alpha adrenergic |
| 11 | channels | | antagonists |
| scaffold of | beta blocker | Cromakilim 19 | potassium |
| atenolol | | | chanel opener |
| 23 | Antidepressant | 26 | Muscarinic |
| | minaprine | | receptor |
| | | 30 | Acetylcholine |
| | | | esterase |
| | | | inhibitor |
| Benzamides | neuroleptic | naphtamide | Affinity for D3 |
| Sulpiride 35 | | 36, 39 | receptor |
| Scaffold | D receptor | 42 | Affinity for D4 |
| clebopride | inhibitor | | receptor, |
| 40 | | | dopaminergic |
| | | | antagonist |
| Thalidomide 44 | sedative | 46 | Inhibitor of |
| | | | TNF |
| Diclofenac 47 | antiinflammatory | 48, 49 | Inhibitor of |
| | | | fibrine TTR |
| | | | amyloid |
| | | | formation |

The numbers indicated in the table 4 refer to the following scaffolds/derivatives

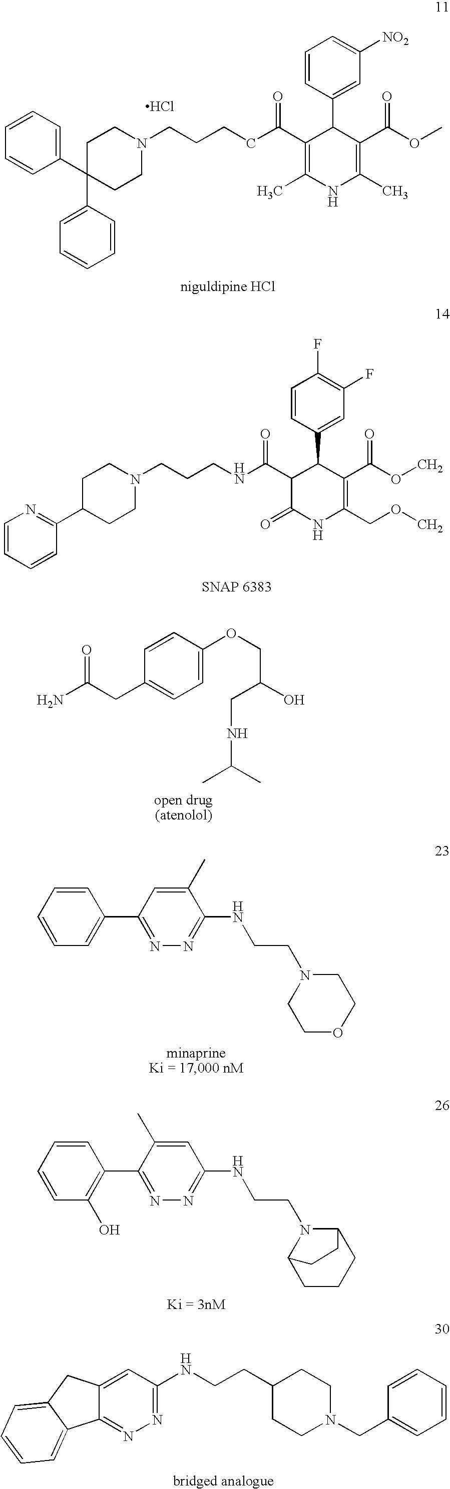

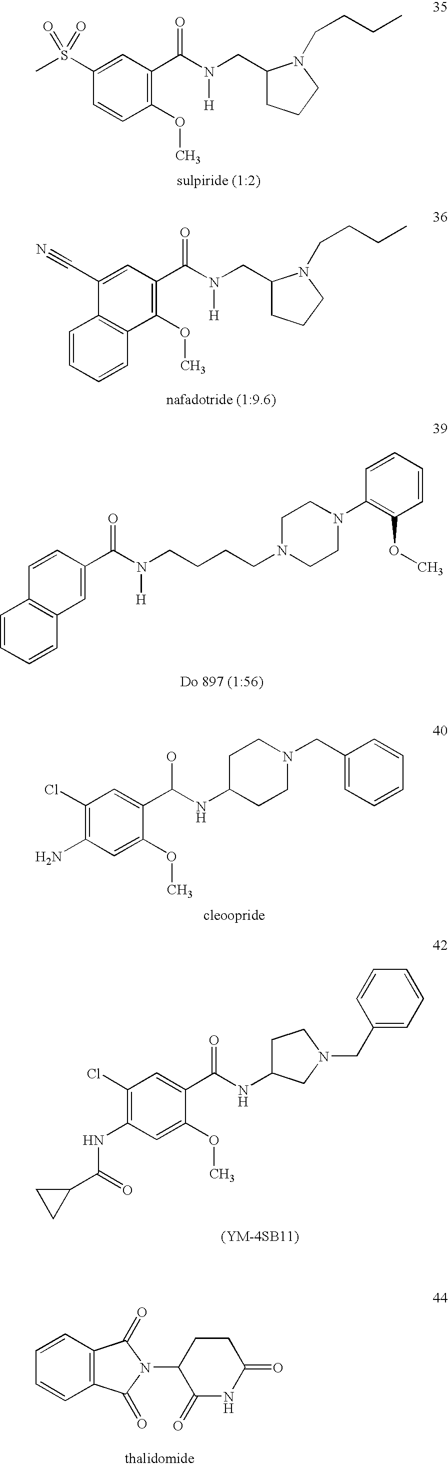

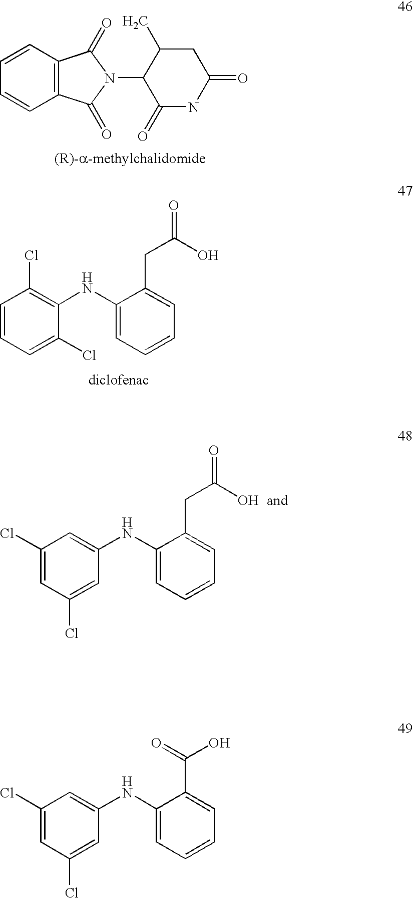

According to an embodiment the SIGNAL entity is a linear or macrocyclic chelate, known in the prior art and well summarized namely in the document WO 01/60416. In particular SIGNAL may be of formula 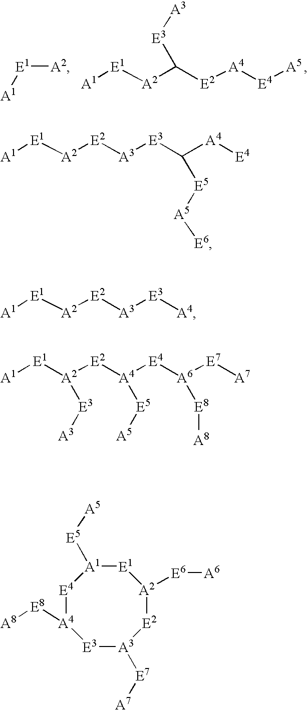
Wherein

A1, A2, A3, A4, A5, A6, A7, and A8 are independently selected at each occurrence from the group: N, NR26,NR19, NR19R20, S, SH,—S(Pg), 0, OH, PR19,PR19R20,-O—P (O) (R21)-O—P(O) R21R22, a bond to the targeting (SCAFFOLD) moiety and a bond to the linking (LINKER) group; Pg is a thiol protecting group;

E1, E2, E3, E4, E5, E6, E7, and E8 are independently a bond, CH, or a spacer group independently selected at each occurrence from the group: C1-C16 alkyl substituted with 0-3 R23, aryl substituted with 0-3R23, C3-10 cycloalkyl substituted with 0-3 R23, heterocyclo-Cyclo alkyl substituted with 0-3 R23, wherein the heterocyclo group is a 5-10 membered heterocyclic ring system containing 1-4 heteroatoms independently selected from N, S, and O, C6-10 aryl-C1-10 alkyl substituted with 0-3 R23, C1-10 cyclo alkyl-C6-10 aryl substituted with 0-3 R23, and a 5-10 membered heterocyclic ring system containing 1-4 heteroatoms independently selected from N, S, and O and substituted with 0-3 R23 R19 and R20 are each independently selected from the group: a bond to the linking group, a bond to the targeting moiety, hydrogen, C1-10 cyclo alkyl substituted with 0-3 R23, aryl substituted with 0-3 R23, C1-10 cycloalkyl substituted with 0-3 R23, heterocyclo-C1-10 alkyl substituted with 0-3 R23, wherein the heterocyclo group is a 5-10 membered heterocyclic ring system containing 1-4 heteroatoms independently selected from N, S, and 0, C6-10 aryl-C1-10 alkyl substituted with 0-3 R23, C1-10 alkyl-C6-10 aryl substituted with 0-3 R23, a 5-10 membered heterocyclic ring system containing 1-4 heteroatoms independently selected from N, S, and O and substituted with 0-3 R23, and an electron, provided that when one of R19 or R20 is an electron, then the other is also an electron;

R21 and R22 are each independently selected from the group: a bond to the linking group, a bond to the targeting moiety, —OH,C1-10 alkyl substituted with 0-3 R23, C1-10 cyclo alkyl substituted with 0-3 R23, aryl substituted with 0-3 R23, C3-10 cycloalkyl substituted with 0-3 R23, heterocyclo-C1-10 alkyl substituted with 0-3 R23, wherein the heterocyclo group is a 5-10 membered heterocyclic ring system containing 1-4 heteroatoms independently selected from N, S, and O, C6-10 aryl-C1-10 alkyl substituted with 0-3 R23, C1-10 alkyl-C6-10 aryl-substituted with 0-3 R23, and a 5-10 membered heterocyclic ring system containing 1-4 heteroatoms independently selected from N, S, and O and substituted with 0-3 R23;

R23 is independently selected at each occurrence from the group: a bond to the linking group, a bond to the targeting moiety, ═O, F, Cl, Br,I,—CF3,—CN,—C02R24,—C (═O) R24, —C(═O) N (R24)2, —CHO,—CH2OR24,—OC(═O) R24, —OC(═O) OR24a, —OR24,—OC(═O) N(R24)2, —NR25C(═O)R24, —NR25C(═O)OR24a, —NR25C(═O)N(R24)2, —NR25SO2N(R24)2, —NR25SO2R24a,—SO3H,SO2R24a-SR24—S (═O) R24a, —SO2N(R24), —N(R24)2, —NHC(═S)NHR24, ═NOR24, N02,—C(═O)NHOR24,—C(═O)NHNR24R24a, —OCH2CO2H,2- (1-morpholino) ethoxy, C1-C5 alkyl, C2-C4 alkenyl, C3-C6 cycloalkyl, C3-C6 cycloalkylmethyl, C2-C6 alkoxyalkyl, aryl substituted with 0-2 R24, and a 5-10 membered heterocyclic ring system containing 1-4 heteroatoms independently selected from N, S, and O;

and wherein at least one of A1, A2,A3, A4,A5, A6, A7,A8 or R23 is a bond to the linking group or targeting moiety;

R24, R24a, and R25 are independently selected at each occurrence from the group: a bond to the linking group, a bond to the targeting moiety, H,C1-C6 alkyl, phenyl, benzyl, C1-C6 alkoxy, halide, nitro, cyano, and trifluoromethyl.

PCTA chelates are also appropriate examples of chelates. It is preferred to prepare chelates which structure is sufficiently easy to produce. Thus it is preferred not to used chelates, namely macrocyclic chelates, that would carry groups having a molecular weight more than 300 or 400. In the formula above, it is preferred to avoid A5E5, A6E6, A7E7,A8E8 with such high molecular weight. The values nil and n2 are appropriate as know in the art, typically between 1 and 10. In particular the chelate is a derivative of DTPA (diethylenetriaminepentaacetic acid) or DOTA (1,4,7,10-tetracyclododécane-N,N′,N″,N′″-tetraacetic acid). Anyway the signal entities and the biovector entities are chemically designed and coupled appropriately.

According to an embodiment, (M) is an ion of a paramagnetic metal of atomic number 21-29, 42-44, or 58-70, namely Gd3+,Mn2+ for the MRI field. In the MRI field the product including the compound (E) has preferably a relaxivity between 4 and 20 mMol-1 s-1 Gd-1 in water; considering that the total relaxivity of the compound may be in the order of at least 100 to 200 mMol-1 s-1 or even more. According to an embodiment (M) is a radionucleide, namely ⁹⁹Tc, ¹¹⁷Sn, ¹¹¹In, ⁹⁷Ru, ⁶⁷ Ga, ⁶⁸Ga, ⁸⁹Zr, ¹⁷⁷Lu, ⁴⁷Sc, ¹⁰⁵Rh; 188Re, ⁶⁰Cu, ⁶² Cu, ⁶⁴Cu, ⁶⁷Cu, ⁹⁰Y, ¹⁵⁹Gd, ¹⁴⁹Pr, ¹⁶⁶Ho. According to an embodiment (M) is a radionucleide for PET imaging.

According to an other embodiment the SIGNAL entity is a metal nanoparticle, typically a superparamagnetic particle called SPIO or USPIO. Preferably the particle is an iron oxide particle, all or some of which are constituted by an iron derivative, generally comprising iron (III), generally an iron oxide or hydroxide. Superparamagnetic particles are normally very small particles of ferrite, including in particular magnetite (Fe₃O₄), maghemite (γ-Fe₂O₃) and other magnetic mineral compounds of transition elements, having a size of less than approximately 100-150 nm. Typically n2 is between 100 and 1000 for one SIGNAL particle. The magnetic particles have a hydrodynamic diameter of from 5 to 300 nm, preferably from 5 to 60 nm, and more preferably from 5 to 30 nm.

According to an other embodiment the signal entity is a lipid nanodroplet containing or not perfluorocarbon (nanoparticulate emulsion) such as those described in WO 03/062198, U.S. Pat. No. 5,958,371, U.S. Pat. No. 5,080,885, U.S. Pat. No. 6,403,056. Such nanodroplets are under the form of an emulsion of nanoparticles that may be coupled to at least 10 000 to 100 000 DTPA for instance. According to an other embodiment the signal entity is a micelle such as described in WO 2004/006965 or a liposome. And more globally, the signal entity may be a system of transport and/or encapsulation of at least a biovector, which contains a part with appropriate hydrophily towards the biovectors; such systems may polymeric particles such as microgel particles, styrene based particles, lipidic multilayers particles, polymers of polysaccharides and ethylene oxide.

The invention also relates to a composition and a contrast product comprising a compound (E) as described above and a diagnostic method comprising its administration to a patient. Examples of appropriate dose, salts, route of administration of such diagnostic compositions for MRI or nuclear medicine are described in many documents such as WO 0226776 incorporated by reference. The diagnostic agents of the invention may be administered to patients for imaging in amounts sufficient to yield the desired contrast with the particular imaging technique. Where the reporter is a metal, generally dosages of from 0.001 to 5.0 mmoles of chelated imaging metal ion per kilogram of patient bodyweight are effective to achieve adequate contrast enhancements. For most MRI applications preferred dosages of imaging metal ion will be in the range of from 0.02 to 1.2 mmoles/kg bodyweight while for X-ray applications dosages of from 0.05 to 2.0 mmoles/kg are generally effective to achieve X-ray attenuation. The compounds according to the invention may be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized. Viewed from a further aspect the invention provides the use of an agent of formula (E) for the manufacture of a contrast medium for use in a method of diagnosis involving administration of said contrast medium to an animate subject and generation of an image of at least part of said subject. Viewed from a still further aspect the invention provides a method of generating an image of an animate human or non-human animal subject involving administering a contrast agent to said subject, e.g. into the vascular system and generating an image of at least a part of said subject to which said contrast agent has distributed, e.g. by Xray, MR, ultrasound, scintigraphy, PET, SPECT, electrical impedance, light or magnetometric imaging modalities, characterised in that as said contrast agent is used an agent of formula (E). Viewed from a further aspect the invention provides a method of monitoring the effect of treatment of a human or non-human animal subject with a drug to combat a condition said method involving administering to said subject an agent of formula (E) and detecting the uptake of said agent by targeted cells or tissues, said administration and detection optionally but preferably being effected repeatedly, e.g. before, during and after treatment with said drug. For instance the drug may target a ligand associated with angiogenesis in tumoral cells, e.g. a cytotoxic agent. Viewed from a yet further aspect the invention provides a process for the preparation of an agent of formula (E), said process comprising the conjugation of a scaffold derivative biovector to a compound detectable in a diagnostic imaging procedure or a chelate compound and if necessary metallating chelant groups in the resultant conjugate with a metal ion detectable in a diagnostic imaging procedure.

The scaffolds to be used in the compounds (E) may act according to several ways. In particular, they may interact with a biological target, the interaction leading to a change of relaxivity of the signal entity compared to the reference, namely by an activation of the complex scaffold-chelate (smart concept), for example due to the enzymatic modification of the scaffold entity, such modification leading a change in the behaviour of the water protons, and thus in an enhanced signal in MRI.

The signal entities may be prepared as described in WO 01/60416 or U.S. Pat. No. 6,221,334 incorporated by reference. The coupling between the signal entities and the scaffolds may be done with linkers such as those described in WO 01/60416 incorporated by reference.

1. Diagnostic compound of formula
(SCAFFOLD)ₙ₁-(LINKER)ₙ₃-(SIGNAL)ₙ₂-(M), (E)
wherein SCAFFOLD is chosen in table 2: 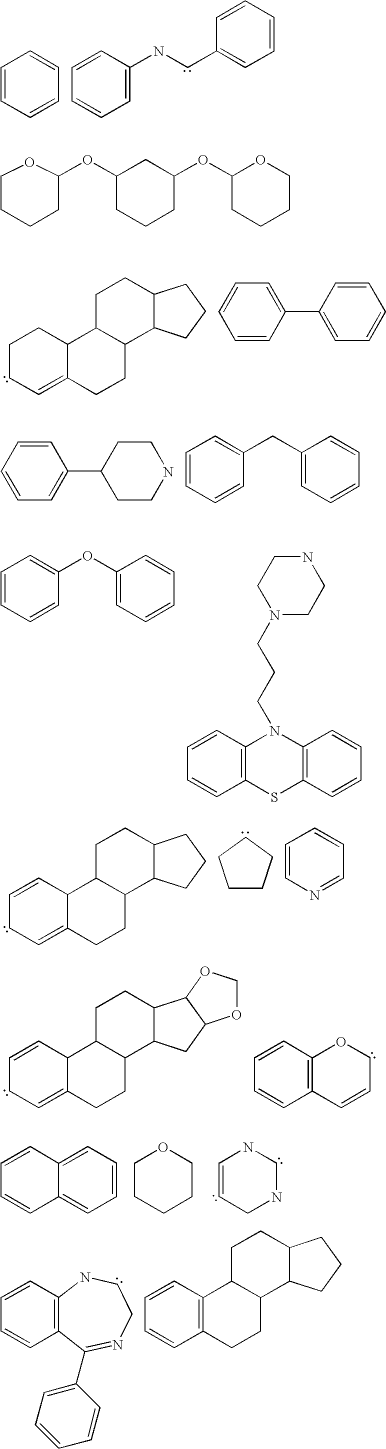 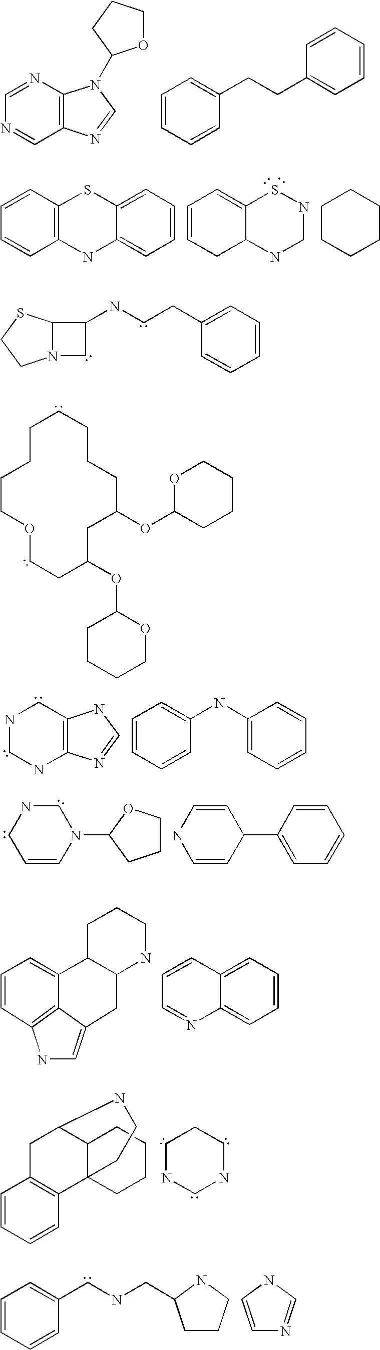 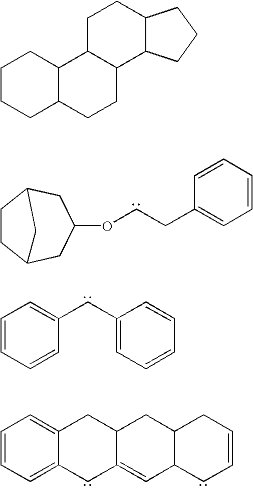
namely Biphenyl; Arylpiperidine; Arylpiperazine; 1,4 dyhydropyridine Dihydropyrimidone; 1,4 benzodiazepine-2-one; 1,5 benzodiazepine-2-one; 1,4-benzodiazepine-2,5 diones; pyrrolo2,1-c 1,4 benzodiazepines 5,11 diones; 1,4 benzothiazepine-5-ones; 5,11-dihydro-benzo pyrido 3,2b 1,4 diazepine-6-ones Benzopyrane; Chromone; Benzopyranone; Coumarine, pyranocoumarine Benzopiperazinones; Quinazolinone; Quinazolindione; Quinoxalinone Imidazoquinoxaline; indole; Benzimidazole Benzofurane Benzothiophene; SIGNAL is a signal entity for medical imaging diagnostic, and pharmaceutically acceptable salts thereof.

2. Compound of claim 1 wherein (M) is an ion of paramagnetic metal of atomic number 21-29, 42-44, or 58-70, preferably Gd3+.

3. Compound of claim 1 exhibiting a relaxivity between 3 and 20 mMol-1s-1 Gd-1 in water.

4. Compound of claim 1 wherein (M) is a diagnostic radionuclide among ⁹⁹ Tc, ¹¹⁷Sn, ¹¹¹In, ⁹⁷Ru, ⁶⁷Ga, ⁶Ga, ⁸⁹Zr, ¹⁷⁷Lu, ⁴⁷Sc, ¹⁰⁵Rh; ¹⁸⁸Re, ⁶⁰Cu, ⁶²Cu, ⁶⁴Cu, ⁶⁷Cu, ⁹⁰Y, ¹⁵⁹ Gd, ¹⁴⁹Pr, ¹⁶⁶Ho.

5. Compound of claim 1 wherein SIGNAL is a metal nanoparticle.

6. Compound of claim 1 wherein SIGNAL is a lipid nanoparticle, micelle, liposome including at least one chelated paramagnetic ion.

7. Composition comprising compounds of claim 1 to 5.

8. Composition comprising an emulsion of nanoparticles wherein said nanoparticles are coupled to at least a scaffold of claim 1.
